(19) United States
(12) Patent Application Publication
(10) Pub. No.: US 2018/0028697 A1
(43) Pub. Date: Feb. 1, 2018

(54) Tongue cleaner that has UV Light to kill bacteria

(71) Applicants: Vinay Jivan, Dublin, CA (US); Anita Jivan, Dublin, CA (US); Paritosh Patel, Pismo Beach, CA (US)

(72) Inventors: Vinay Jivan, Dublin, CA (US); Anita Jivan, Dublin, CA (US); Paritosh Patel, Pismo Beach, CA (US)

(21) Appl. No.: 15/225,648

(22) Filed: Aug. 1, 2016

(51) Int. Cl. A61L 2/00; B08B 1/00

(52) U.S. Cl. CPC A61L 2/0047; B08B 1/005; A61L 2202/11; A61L 2202/17

(57) ABSTRACT

A tongue cleaner that can be used by an operator or an individual that is capable of removing debris and killing bacteria from the tongue. The improvement comprises of a UV light source located on the tongue cleaner scraper head that would let UV light shine directly onto the tongue, as it is stroked against the tongue, it would kill the germs and bacteria.

### FIELD OF THE INVENTION

This invention would help maintain good oral hygiene by cleaning the surface of your tongue and also killing bacteria and germs that causes bad breath. Particularly, this invention relates to a tongue scraper that safely removes plaque, kills bacteria and debris buildup from tongue surface, which is much better than just brushing or scraping the tongue.

### BACKGROUND OF THE INVENTION

UV light is an effective way to kill germs and bacteria. The major reason for bad breath is plaque, bacteria and debris in our mouths.

Toothbrushes and tongue scrapers help remove plaque and debris from the tongue but do a poor job in killing the bacteria that resides on your tongue. Conventional toothbrushes with bristles are often uncomfortable to use on the tongue and hence many people don't use it to clean their tongue. Therefore, there is an unmet need that toothbrushes and tongue scrapers need to overcome in order to enhance oral hygiene.

### SUMMARY OF THE INVENTION

This invention centers around a unique design of a tongue scraper that can deliver UV light, and at the same time allows for optimum removal of plaque and debris from the tongue.

The present implementation includes a head that forms the scraper edge, that also directs the UV light to the surface of the tongue and a handle that houses the power source for the UV-Light. The UV Light source would typically be a UV LED that would directly be mounted inside the head or in the handle. When mounted in the handle the head would use built in light pipes (refraction properties) to direct the UV light to the tongue. The handle would house the batteries, or even chargeable batteries that could be charged wireless using induction charging or with the use of solar cells.

### BRIEF DESCRIPTION OF DRAWINGS

FIG. 1: Is a side view of the tongue cleaner in accordance with and/or use with the present invention.

FIG. 2: Is a cross sectional view from the bottom showing all the major components of the design. The handle has the battery, the electrical conduits, the control button and optional electronics. This view also shows the bottom of the head showing the beveled scraper edge, the light diffuser/lens and the UV LED placed behind the light diffuser/lens.

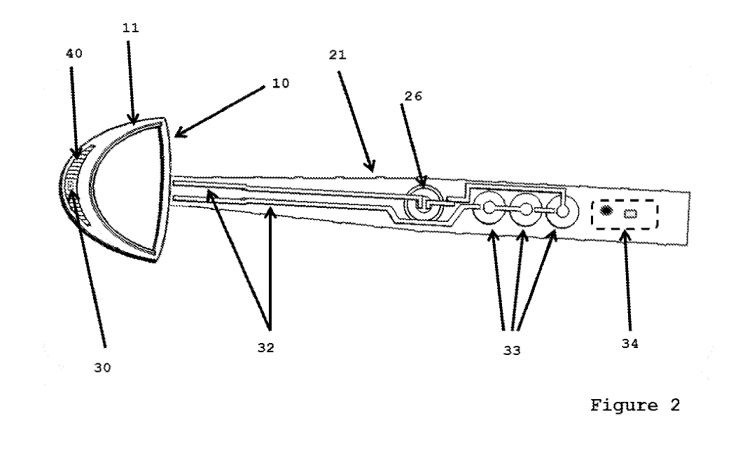

FIG. 3: Shows the UV Light being part of the handle and that the handle and head as being two separate parts. In this case the head part has no electrical components, it only has light pipes to guide the light to the desired location. The handle and head connect together via a quick snap connection that is keyed to ensure correct orientation of the head to the handle.

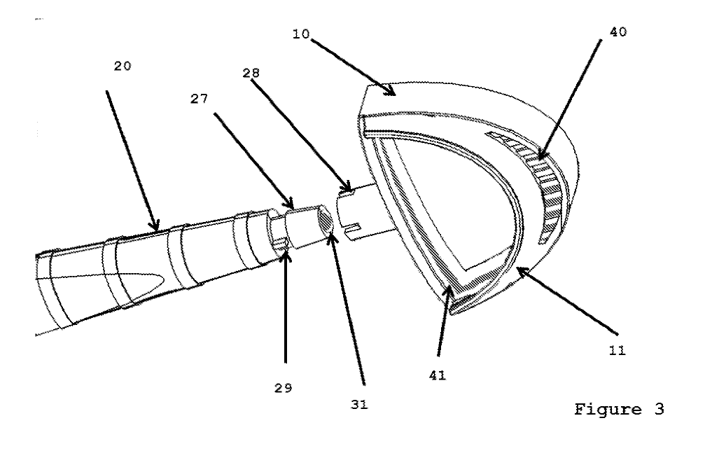

FIG. 4: Is a view from the top. The UV light is not visible from the top. The handle has the control button and also show an optional LCD display.

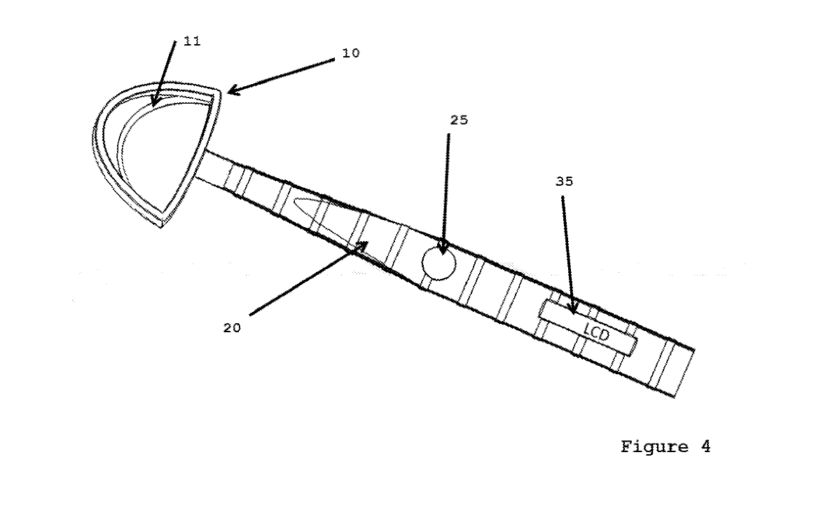

### REFERENCE NUMERALS IN DRAWINGS

10 Head part of cleaner
11 Beveled scraper edge
20 Handle part of scraper
21 Cross sectional view of handle from bottom
25 Control button to turn on/off UV light
26 Cross section view of control button from bottom
27 Quick connect plug
28 Quick connect receptacle
29 Key to align head and handle
30 UV Led mounted in head
31 UV Led mounted in handle
32 Built in electrical conduits to carry power
33 Batteries to power UV Light source
34 Optional electronics to control UV Light, charging the batteries, Blue-tooth or Wi-Fi
35 Optional LCD display for status indicator
40 Light diffuser/lens to distribute UV Light evenly
41 Light pipes built inside head

### DETAILS OF THE INVENTION

The tongue cleaner includes a head (10) that forms the scraper which will channel up the plaque and debris. In close proximity to the scraper's edge (11) is a parallel light diffuser/lens (40) that will shine UV Light onto the surface of the tongue, killing all the bacteria and germs in its path.

The handle (20) and the head (10) are integrally formed, with the handle (20) designed to house the power for the UV Light. The handle would have the batteries (33) and if chargeable batteries are used charging could be done via induction or solar cells hence eliminating the need for wires for charging. The handle (20) could also house a go button (25) that the user/operator would press to control the UV Light. Simple controls such as ON/OFF, intensity control, strobing and timer features could all be controlled using this button (25) and some electronics/microcontroller (34) could also be housed inside the handle to provide Bluetooth, Wi-Fi, LCD and charging features.

As this tongue cleaner is stroked against one tongue, in a single stroke it would not only remove all the plaque and debris from the surface of the tongue, but at the same time it would kill the germs and bacteria in the UV Light path.

Due to cost and other benefits one could also have the UV Light source housed inside the handle (31). In this case the scraper head (10) would use clear and opaque plastic to direct the UV light to the tongue surface. The scraper head would hence act as a light pipe (41) for the UV Light.

With the UV Light source (31) built inside the handle (20) the head (10) and the handle (20) would be two separate parts that would snap together (28), (29). The scraper head (10) could be made to be disposable, this would allow for dentists, oral hygienists and other individuals to quickly change and dispose of the used scraper head for a new device. Since the handle (20) part won't be disposed of it could be made to have some optional features, such as wireless charging, an LCD (35) to provide direct user feedback of UV light settings, battery level and usage. It could also have Bluetooth or Wi-Fi connectivity allowing for data usage to be down loaded to a smart device.

1. A tongue cleaner that can be used by an operator or individual that is capable removing debris and plaque, and is also capable of killing the bacteria and germs on your tongue by using UV light. The tongue cleaner comprising of:
A head (10) that has a light diffuser/lens and a beveled edge, which can be made from plastic, metal or rubber that forms the scraper edge (11).
A handle (20) that houses all the electrical conduits, the batteries (33), the control circuitry and optional electronics (34) that would power the UV Light source.
The UV light source (30) can be mounted directly in the head (10) behind the light diffuser/lens (40) or can be mounted in the handle (20). When mounted in the handle the head would use built in plastic light pipes to direct the light to the desired position.
The UV Light source can be from a UV LED or any UV Lamp source that can be small enough to mount inside the handle (20) or head (10).

2. The tongue cleaner of claim 1, control of the UV Light is made possible by having a control button (25) built into the handle to accommodate single handed operation of the tongue cleaner. The control button (25) would not only control the UV light being ON/OFF but could also control the intensity of the light by selecting a pre-programmed choice, these control features are also part of the claim.

3. The tongue cleaner of claim 1, use of light pipes (41) to direct UV light from the handle (20) to the head (10) and to have the UV light shine directly to the surface of the tongue are also part of the claim.

4. The tongue cleaner of claim 1, having the handle (20) and head (10) being two separate devices that snap together and after use the head being disposed and replaced with a clean new one are part of this claim.

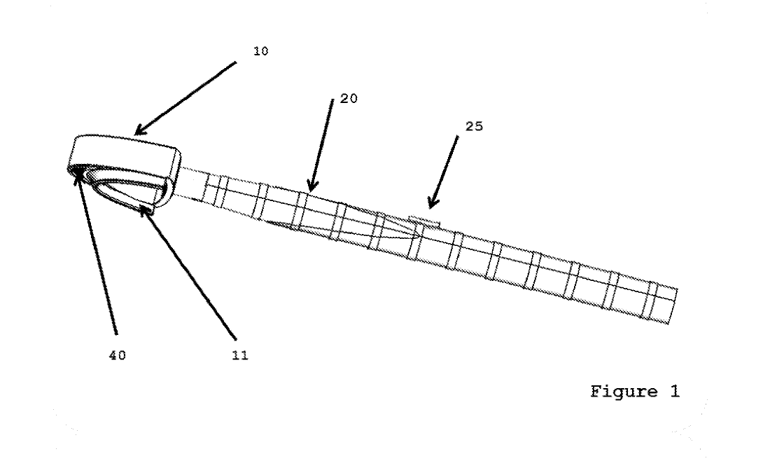

5. FIG. 1 shows an example of the tongue cleaner claim 1. Other possible shapes for the head (10), scraper edge (11), the handle (20) and position of the UV Light sources that shine directly at the surface of the tongue are all part of this claim.
